(19) United States
(12) Patent Application Publication
(10) Pub. No.: US 2012/0109255 A1
(43) Pub. Date: May 3, 2012

(54) Retina Stimulation Apparatus and Manufacturing Method Thereof

(75) Inventor: Long-Sheng Fan, Hsinchu (TW)

(73) Assignee: NATIONAL TSING HUA UNIVERSITY, HSINCHU (TW)

(21) Appl. No.: 13/102,596

(22) Filed: May 6, 2011

Related U.S. Application Data
(60) Provisional application No. 61/407,229, filed on Oct. 27, 2010.

(51) Int. Cl. A61F 9/08; H05K 3/10; A61N 1/36

(52) U.S. Cl. 607/54; 29/846

(57) ABSTRACT

The invention discloses a retina stimulation apparatus and a manufacturing method thereof The apparatus comprises a pixel unit, a power supply module and a flexible package. The pixel unit and power supply module are disposed on and covered by the flexible package, and the power supply module can supply power to the pixel unit after being charged. Each pixel unit comprises a photosensor, a signal processing and driving unit and a stimulating electrode. The photosensor detects an incident light and provides a sensing signal to the signal processing and driving unit, and the processing unit generates a stimulation signal with an appropriate waveform to the stimulating electrode according to the sensing signal. Through the stimulating electrode, a stimulation current is used to stimulate retina ganglion cells.

### CROSS-REFERENCE TO RELATED APPLICATION

This application claims the benefit of U.S. provisional application Ser. No. 61/407,229, filed Oct. 27, 2010, which is incorporated herein by reference in its entirety.

### BACKGROUND OF THE INVENTION

1. Field of the Invention

The present invention relates to a retina stimulation apparatus and its manufacturing method, and more particularly to a retina stimulation apparatus and a manufacturing method of the retina stimulation apparatus capable of recovering from blindness caused by an age-related macular disease.

2. Description of the Related Art

At present, a common retina stimulating apparatus as disclosed in U.S. Pat. No. 7,158,836 includes a plurality of electrode units installed on a support plane, and a plurality of pins directly installed and fixed onto a sclera of the support plane. Since this conventional retina stimulating apparatus cannot be attached completely onto the curvature of eyeballs, a critical value of non-uniform stimulations is resulted and a portion of electrodes requires a relatively large current to stimulate the retina. As a result, the requirements for a high-resolution array, high efficiency and low power cannot be achieved.

In addition, another retina stimulating apparatus as disclosed in U.S. Pat. No. 7,035,692 lacks the features of integrating a light sensing element (CMOS), a driving device and a stimulating electrode with one another, and has a poor effect of stimulating the retina.

Therefore, it is an important subject for designers and manufacturers of the related industry to provide a retina stimulation apparatus capable of matching the eyeball curvature and integrating each component.

### BRIEF SUMMARY

Therefore, it is a primary objective of the present invention to overcome the aforementioned shortcomings of the prior art by providing a retina stimulation apparatus and its manufacturing method, and the retina stimulation apparatus is matched and attached onto the curvature of eyeballs, and each component is integrated onto the flexible package, such that the problem of damaging other cells caused by an excessive power consumption of the conventional retina stimulation apparatus can be overcome.

To achieve the foregoing objective, the present invention provides a retina stimulation apparatus comprising a plurality of pixel units and a flexible package. The flexible package is provided for carrying the pixel units and the power supply module, and each pixel unit includes a photosensor, a signal processing and driving unit and a stimulating electrode. The photosensor is provided for sensing an incident light and generating a sensing signal, and the signal processing and driving unit is provided for receiving and processing the sensing signal to generate an electric stimulation waveform, and the stimulating electrode is provided for receiving the electric stimulation waveform to generate a corresponding stimulation current to stimulate a retinal cell.

The retina stimulation apparatus is installed on a ganglion cell, and the photosensor and the signal processing and driving unit are installed on the other side without the stimulating electrode on a middle layer.

The retina stimulation apparatus is installed on a ganglion cell, and the photosensor and the signal processing and driving unit are installed on the same side of the stimulating electrode on a middle layer.

The retina stimulation apparatus is installed between a bipolar cell and a rod and cone, and the photosensor and the signal processing and driving unit are installed on the other side of the stimulating electrode on a middle layer.

The retina stimulation apparatus is installed between a bipolar cell and rods and cones, and the photosensor and the signal processing and driving unit are installed on the same side of the stimulating electrode on a middle layer.

The retina stimulation apparatus further comprises a power supply module coupled to the pixel unit, and the power supply module supplies electric power to the pixel units after the power supply module is charged via a wireless charging process.

A perforation hole is formed between two pixel units and used for allowing air or tissue liquids to flow through both upper and lower sides of the retina stimulation apparatus.

Each signal processing and driving unit is electrically coupled by a conductive wire and used for exchanging each sensing signal to adjust the intensity of a background light.

Each stimulating electrode includes a guard ring installed under each stimulating electrode for regionally stimulating the retinal cell.

Each signal processing and driving unit further includes a sensing circuit for detecting the mode of a retinal cell, and each signal processing and driving unit is provided for controlling the stimulating electrode to stimulate the retinal cell according to the mode of the retinal cell.

The stimulating electrode is in a convex umbrella shape.

Each convex umbrella-shaped stimulating electrode includes a plurality of protrusions disposed on the same plane.

The retina stimulation apparatus further comprises a remote control device coupled to the pixel units via a wireless communication and provided for controlling the pixel units from a long distance.

The wireless communication for coupling the remote control device with the pixel units includes an optical communication, a radio frequency communication or a wireless communication.

The flexible package is made of a biocompatible material.

The biocompatible material is polyimide, poly(dimethyl siloxane) (PDMS) or parylene.

To achieve the objective of the present invention, the invention further provides a manufacturing method of a retina stimulation apparatus, and the method comprises the steps of: providing a substrate; integrating a plurality of signal processing and driving units, a plurality of photosensors and a plurality of stimulating electrodes on the substrate to form a plurality of pixel units; forming an electrically conductive layer on the stimulating electrode and a first barrier layer on the other area of the pixel unit; forming a first biocompatible material layer on the first barrier layer and a first holding substrate on the first barrier layer; removing the substrate to expose the signal processing and driving unit and the photosensor; forming a second barrier layer on the exposed signal processing and driving unit and photosensor; forming a second biocompatible material layer on the second barrier layer after a perforation hole is formed between two pixel units, and covering the first biocompatible material layer and the second biocompatible material layer onto each pixel unit; forming a second holding substrate on the second biocompatible material layer, and removing a portion of the first biocompatible material layer to expose the electrically conductive layer from the stimulating electrode; and finally removing the second holding substrate.

This method further comprises the step of electrically coupling a conductive wire with each signal processing and driving unit to exchange each sensing signal to adjust the intensity of a background light.

This method further comprises a power supply module coupled to the pixel units for supplying electric power to the pixel units after the power supply module is charged.

The photosensor is provided for sensing an incident light and generating a sensing signal, and the signal processing and driving unit is provided for receiving and processing the sensing signal to generate a corresponding electric stimulation waveform, and the stimulating electrode is provided for receiving the electric stimulation waveform to generate a corresponding stimulation current to stimulate a retinal cell.

This method further comprises the step of installing a guard ring under each stimulating electrode for regionally stimulating each retinal cell.

This method further comprises a sensing circuit installed in each signal processing and driving unit, for detecting the mode of a retinal cell, and each signal processing and driving unit is provided for controlling the stimulating electrode to stimulate the retinal cell according to the mode of the retinal cell.

The stimulating electrode is in a convex umbrella shape.

Each convex umbrella-shaped stimulating electrode includes a plurality of protrusions not disposed on the same plane.

This method further comprises a remote control device coupled to the pixel units by a wireless communication and provided for controlling the pixel units from a long distance.

The wireless way of coupling the remote control device with the pixel units includes an optical communication, a radio frequency communication or a wireless communication.

The first biocompatible material layer and the second biocompatible material layer are made of a flexible material.

The flexible material is polyimide, poly(dimethyl siloxane) (PDMS) or parylene.

The first barrier layer and the second barrier layer are made of a silicon carbide (SiC) or a diamond like carbon film (DLC Film).

In summation of the above, the retina stimulation apparatus and its manufacturing method of the present invention have one or more of the following advantages:

(1) In the retina stimulation apparatus and its manufacturing method of the present invention, the retina stimulation apparatus can be surface mounted onto an eyeball on the flexible substrate to reduce the current for stimulating the retina and avoiding damages to the cells.

(2) In the retina stimulation apparatus and its manufacturing method of the present invention, the photosensors, driving devices and stimulating electrodes are integrated in a pixel unit to overcome the problem of having the poor sensing and stimulating effects.

### BRIEF DESCRIPTION OF THE DRAWINGS

FIG. 1 is a flow chart of a manufacturing method of a retina stimulation apparatus in accordance with the present invention;

FIGS. 2A-2E are schematic views of a manufacturing method of a retina stimulation apparatus in accordance with a preferred embodiment of the present invention;

FIG. 3A is a schematic view of a retina stimulation apparatus of the present invention;

FIG. 3B is a schematic view of a pixel unit in accordance with a preferred embodiment of the present invention;

FIG. 4A is a schematic view of a guard ring of a retina stimulation apparatus in accordance with the present invention;

FIG. 4B is a cross-sectional view of a guard ring of a retina stimulation apparatus in accordance with the present invention;

FIG. 5A is a schematic view of an epi-retina stimulation apparatus in accordance with a first preferred embodiment of the present invention;

FIG. 5B is a schematic view of an epi-retina stimulation apparatus in accordance with a second preferred embodiment of the present invention;

FIG. 5C is a schematic view of a sub-retina stimulation apparatus in accordance with a first preferred embodiment of the present invention;

FIG. 5D is a schematic view of a sub-retina stimulation apparatus in accordance with a second preferred embodiment of the present invention;

FIG. 6 is a schematic view of installing a plurality of retina stimulation apparatuses of the present invention;

FIG. 7 is a graph of a stimulation current versus a distance of a retina from a stimulation electrode of a retina stimulation apparatus of the present invention;

FIG. 8A is a schematic view of an epi-retina stimulation apparatus in accordance with a third preferred embodiment of the present invention;

FIG. 8B is another is a schematic view of an epi-retina stimulation apparatus in accordance with a third preferred embodiment of the present invention;

FIG. 9A is a schematic view of adjusting the brightness of a background light by a signal processing and driving unit of the present invention;

FIG. 9B is a circuit diagram of adjusting the brightness of a background light by a signal processing and driving unit of the present invention;

FIG. 9C is a schematic view of reducing background noises by a signal processing and driving unit of the present invention;

FIG. 10A is a schematic view of an epi-retina stimulation apparatus in accordance with a fourth preferred embodiment of the present invention; and

FIG. 10B is a schematic view of an epi-retina stimulation apparatus in accordance with a fifth preferred embodiment of the present invention.

### DETAILED DESCRIPTION

The foregoing and other objectives, characteristics and advantages of the present invention will become apparent by the detailed description of a preferred embodiment as follows. It is noteworthy to point out that each preferred embodiment is provided for the purpose of illustrating the present invention only, but not intended for limiting the scope of the patent claims.

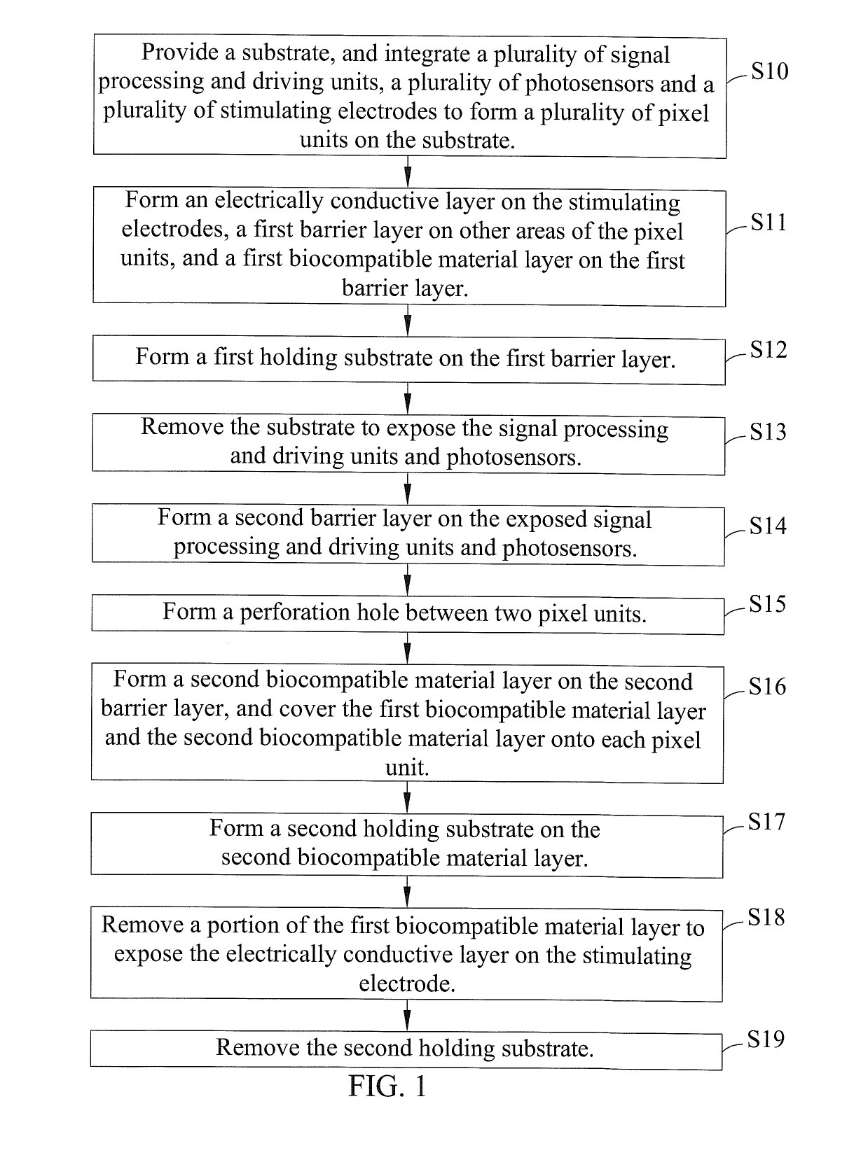

With reference to FIG. 1 for a flow chart of a manufacturing method of a retina stimulation apparatus in accordance with the present invention, the manufacturing method comprising the following steps:

(S10): Provide a substrate, and integrate a plurality of signal processing and driving units, a plurality of photosensors and a plurality of stimulating electrodes to form a plurality of pixel units on the substrate.

(S11) Form an electrically conductive layer on the stimulating electrodes, a first barrier layer on other areas of the pixel units, and a first biocompatible material layer on the first barrier layer.

(S12) Form a first holding substrate on the first barrier layer.

(S13) Remove the substrate to expose the signal processing and driving units and photosensors.

(S14) Form a second barrier layer on the exposed signal processing and driving units and photosensors.

(S15) Form a perforation hole between two pixel units.

(S16) Form a second biocompatible material layer on the second barrier layer, and cover the first biocompatible material layer and the second biocompatible material layer onto each pixel unit.

(S17) Form a second holding substrate on the second biocompatible material layer.

(S18) Remove a portion of the first biocompatible material layer to expose the electrically conductive layer on the stimulating electrode.

(S19) Finally, remove the second holding substrate.

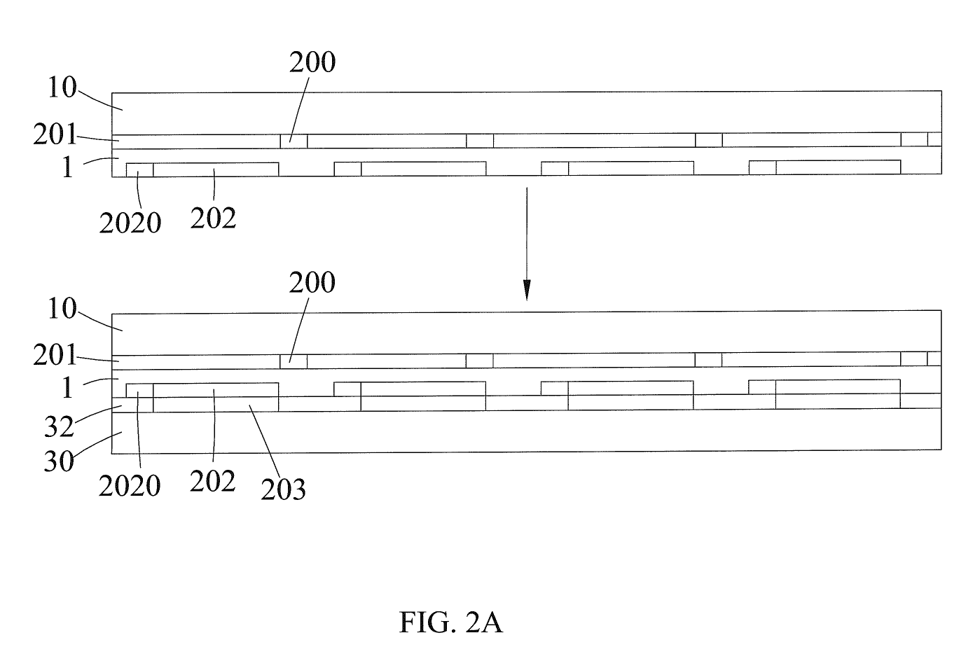

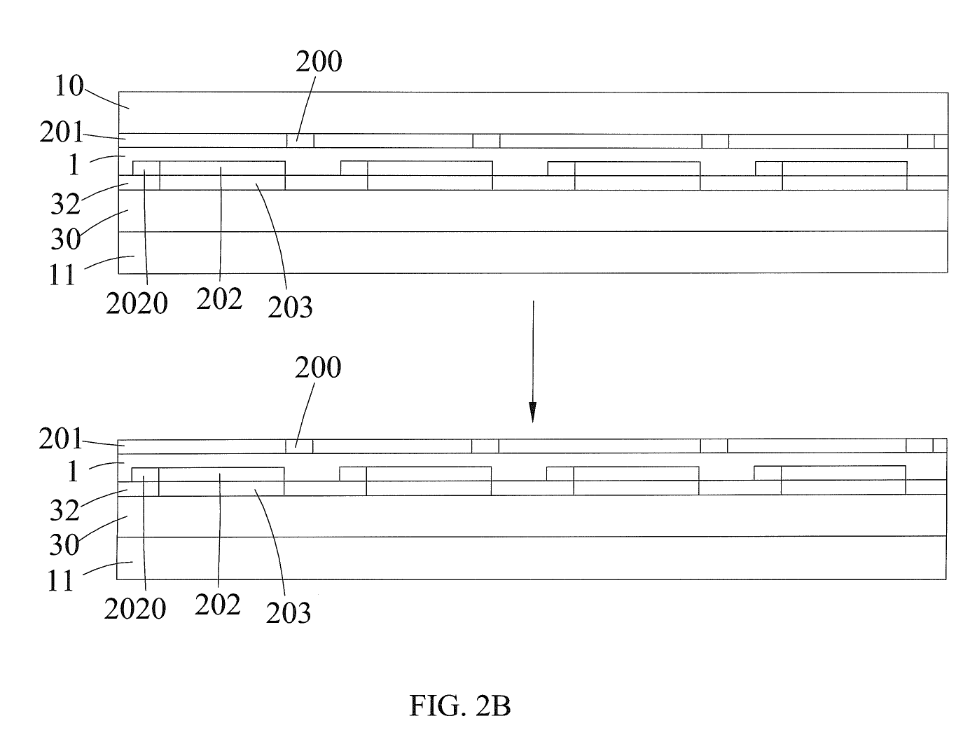

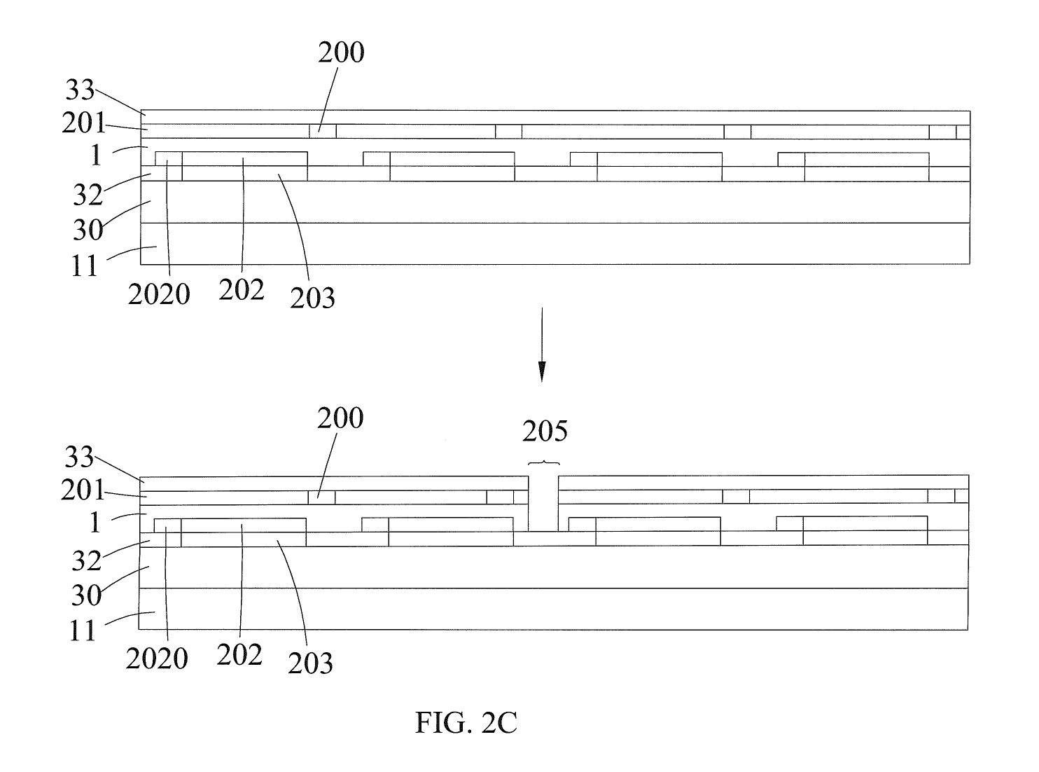

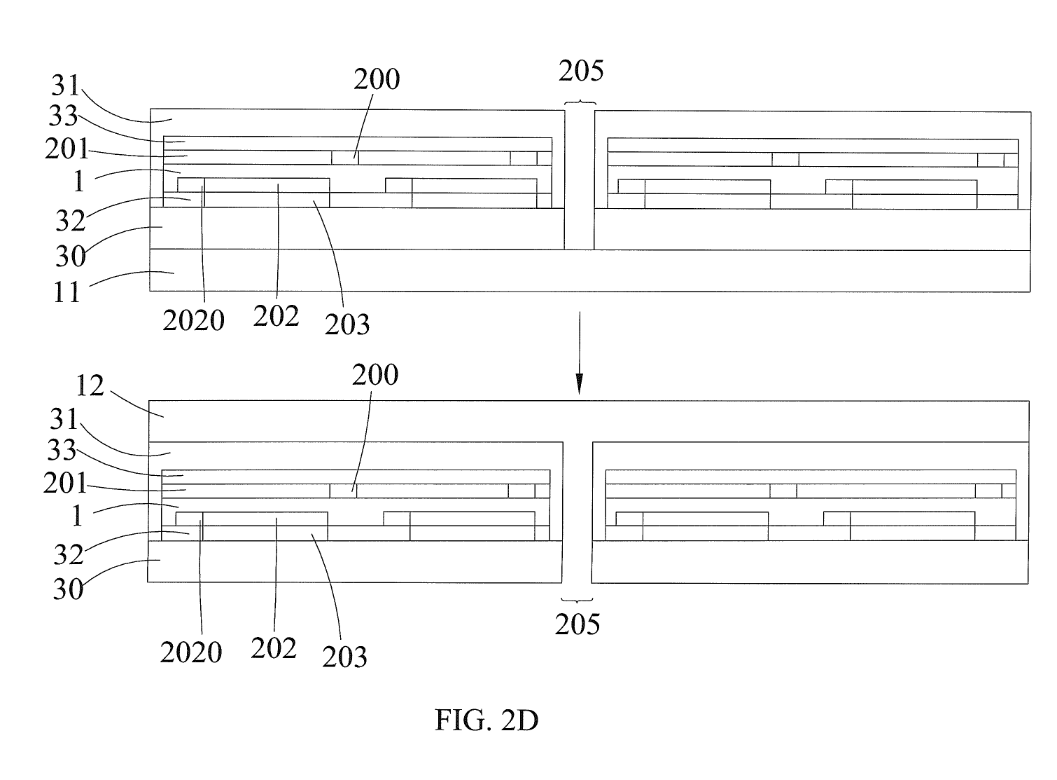

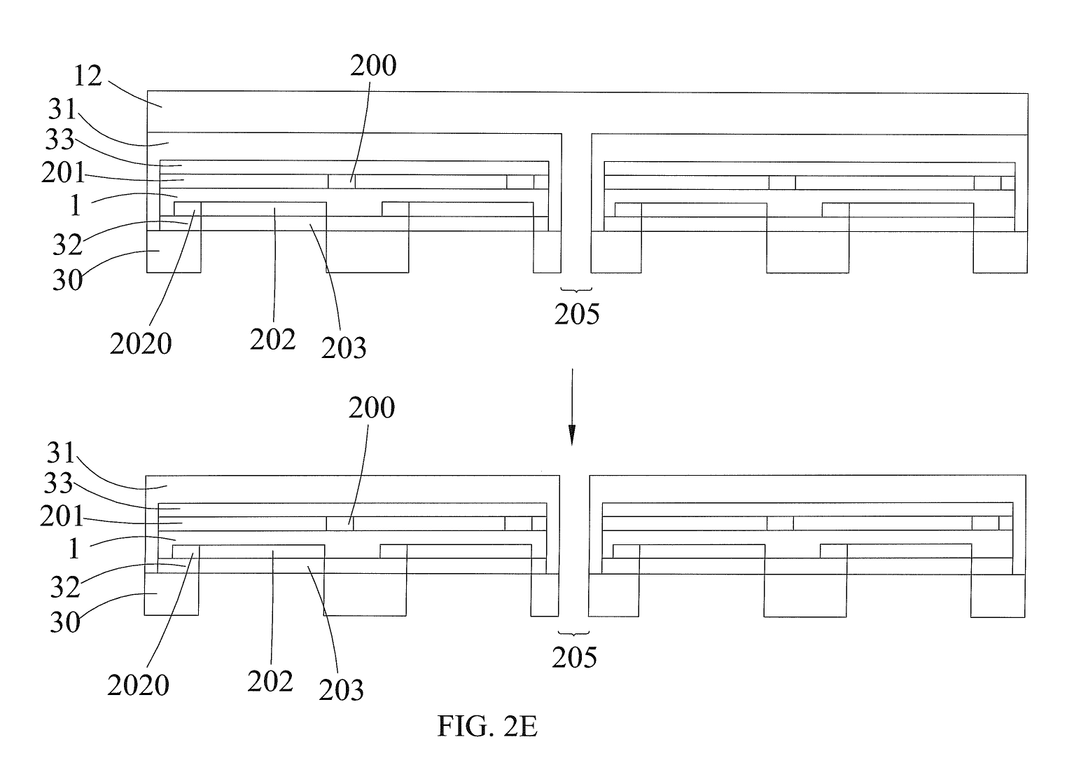

With simultaneous reference to FIGS. 2A-2E for schematic views of a manufacturing method of a retina stimulation apparatus in accordance with a preferred embodiment of the present invention, the manufacturing method comprises the following steps:

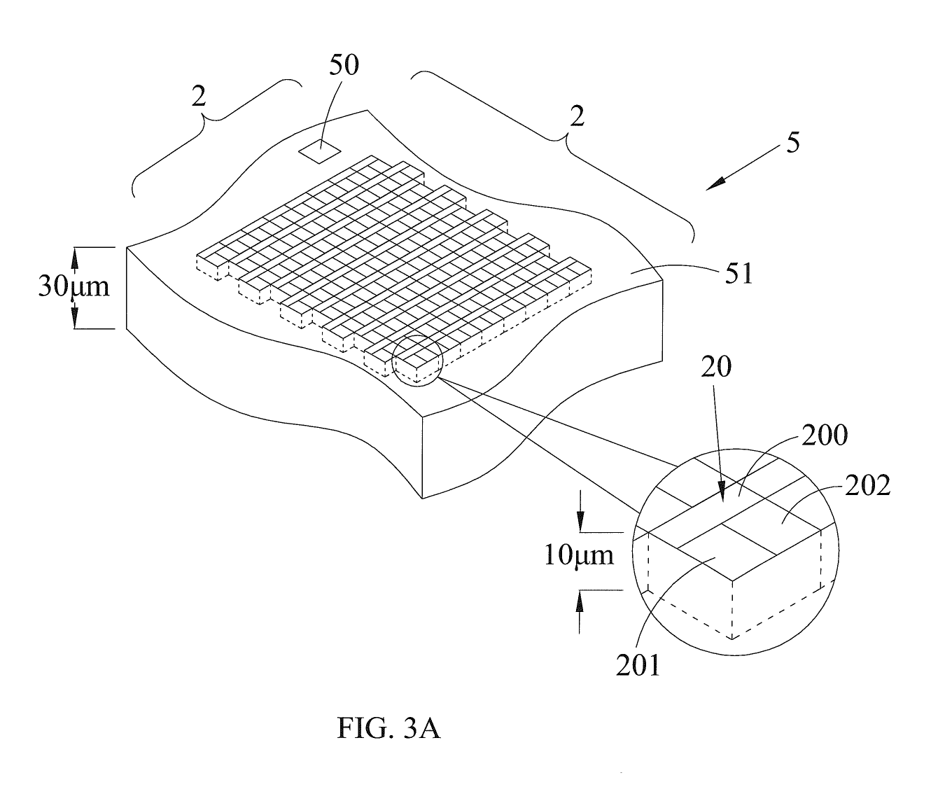

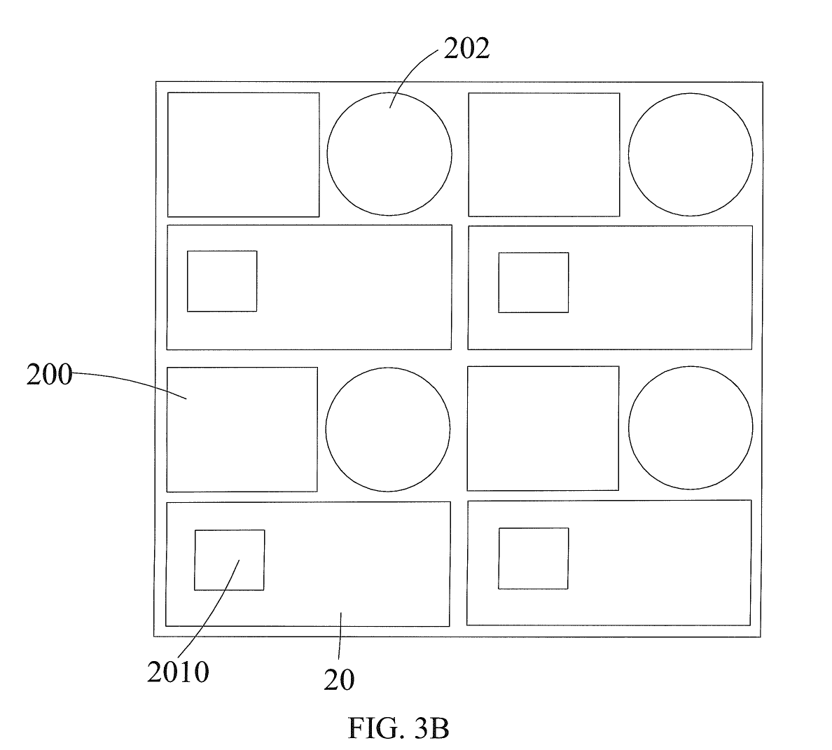

(S10) Integrate a plurality of signal processing and driving units 201, a plurality of photosensors 200 and a plurality of stimulating electrodes 202 to form a plurality of pixel units 20 (shown in FIG. 3A) on a substrate 10. In this step, the integration can be achieved by applying a standard or slightly modified CMOS semiconductor manufacturing process or CMOS image sensor (CIS) manufacturing process on a silicon wafer (substrate 10). In one preferred embodiment, the photosensors include but not limited to a PN Junction or being treated by an appropriate doping process, wherein the stimulating electrodes 202 are covered by a material including but not limited to a titanium-nickel alloy, and the stimulating electrodes 202 are finally exposed.

Step (S11): Form an electrically conductive layer 203 on the stimulating electrode 202, and the electrically conductive layer 203 is made of a material including but not limited to iridium oxide (IrOx), platinum (Pt), titanium nitride (TiN) or iron oxide (FeOx) for providing a better interface between the stimulating electrode 202 and a first biocompatible material layer 30. The first biocompatible material layer 30 and second biocompatible material layer 31 are made of a material including but not limited to polyimide, poly(dimethyl siloxane) (PDMS) or parylene. In addition, the first barrier layer 32 and second barrier layer 33 installed on other areas of the pixel unit 20 are made of a material including but not limited to silicon carbide (SiC) or diamond like carbon film (DLC film), and the location above the stimulating electrode 202 is bored by a semiconductor lithographic process for being covered with the electrically conductive layer 203.

Step (S 12): A first holding substrate 11 is applied for being held by hand or clamped by a machine for thinning or removing the substrate 10 in Step (S13), and the thinning and removing processes can be a combination of a grinding process and an etching process, but the present invention is not limited to such arrangement only. The substrate 10 may not be removed, but it is just reduced to a thickness approximately equal to several tens of microns that allows a light to be detected by the photosensors 200, and that the whole substrate 10 is flexible.

After the second barrier layer 33 is formed on the exposed signal processing and driving units 201 and photosensors 202, a perforation hole 205 is formed between two pixel units 20 and provided for allowing tissue liquids in a human body to pass through both sides of the retina stimulation apparatus, and this boring process is carried out by a lithographic etching procedure. After the second biocompatible material layer 31 is covered onto the perforation hole 205, the process of boring the perforation hole 205 still can be preformed, such that the whole pixel unit 20 can be covered by the first and second biocompatible material layer, and just the perforation hole 205 is exposed for exchanging tissue liquids.

Step (S17), the second holding substrate 12 is disposed on the second biocompatible material layer 31, and is used in Step (S18) so that the whole retina stimulation apparatus is held by hand or clamping it by a machine for removing a portion of the first biocompatible material layer 30 to expose the electrically conductive layer 203 on the stimulating electrode 202 in order to stimulate retinal cells. Since the total thickness of the first biocompatible material layer 30 and second biocompatible material layer 31 is very small, therefore the total thickness of the whole retina stimulation apparatus is also very small (approximately equal to 30 μm) to allow light to pass therethrough.

With reference to FIG. 3A for a schematic view of a retina stimulation apparatus of the present invention, the retina stimulation apparatus 5 comprises a pixel array 2, a power supply module 50 and a flexible package 51. The flexible package 51, which has a thickness of, preferably but not limited to, 30 μm, is for carrying and covering the pixel array 2 and the power supply module 50, and the power supply module 50 is provided for supplying electric power to the pixel array 2 after the power supply module 50 is changed wirelessly. The pixel array 2 comprises a plurality of pixel units 20, having a thickness of, preferably but not limited to, 10 μm, and each pixel unit 20 includes a photosensor 200, a signal processing and driving unit 201 and a stimulating electrode 202. The photosensor 200 is provided for sensing an incident light to generate a sensing signal, and the signal processing and driving unit 201 is provided for receiving and processing the sensing signal to generate an electric stimulation waveform, and the stimulating electrode 202 is provided for receiving the electric stimulation waveform to generate a stimulation current to stimulate a retinal cell.

With reference to FIG. 3B, the signal processing and driving unit 201 further includes a sensing circuit 2010 preferably coupled to the stimulating electrode 202 or the sensing circuit 2010 is capable of detecting the mode of retinal cell independently, and each signal processing and driving unit 201 controls the stimulating electrode 202 to stimulate the retinal cell according to the mode of the retinal cell. In one preferred embodiment, the sensing circuit 2010 is at a correction mode for detecting the response time of the retinal cell to determine whether the mode of the retinal cell is an ON cell or an OFF cell. The signal processing and driving unit 201 further determines the stimulation mode of the retinal cell with respect to the specific stimulating electrode 202 according to the response time of the retinal cell.

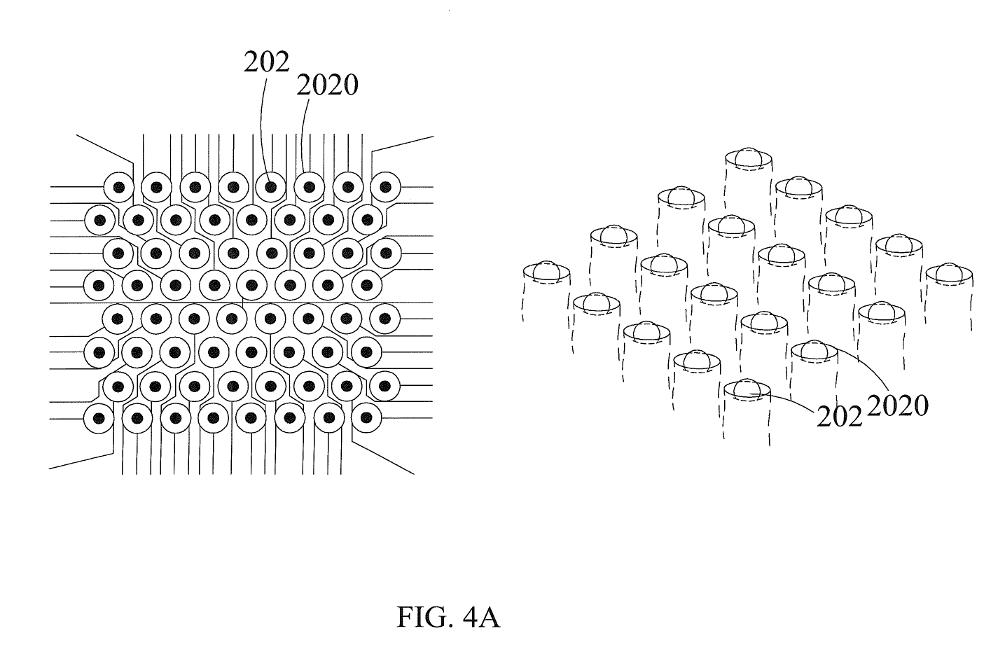

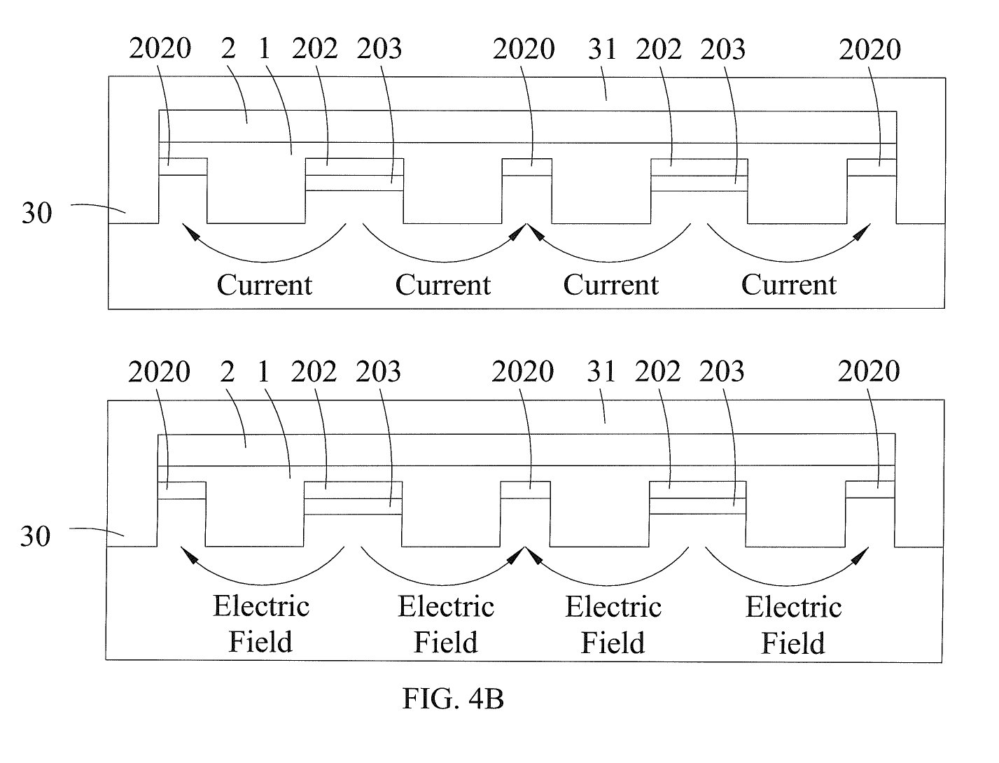

With reference to FIGS. 4A and 4B, a guard ring 2020 is further installed around the periphery under each stimulating electrode, and the guard ring 2020 is a local-area reference electrode serving as a current return path (under a current driving mode) or an electric field return path (under a voltage driving mode). The guard rings 2020 are used for providing a path for the stimulation current or electric field, so that the stimulation current will not stimulate cells far from the stimulating electrode to achieve the effects of regionally stimulating eye cells while protecting other cells.

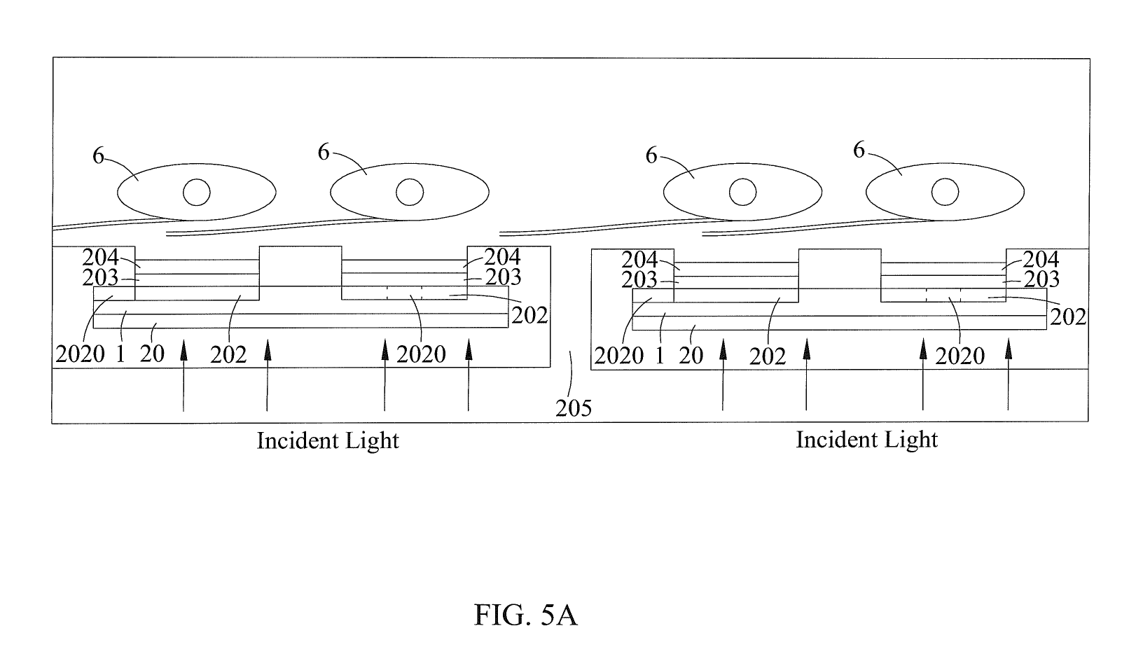

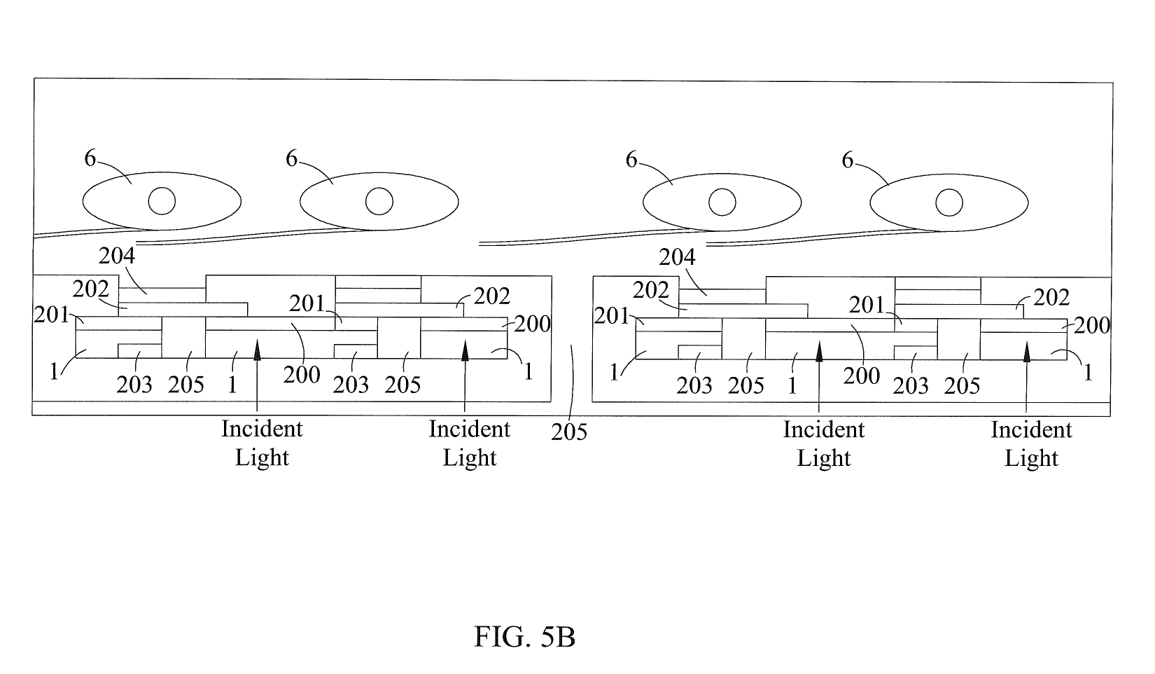

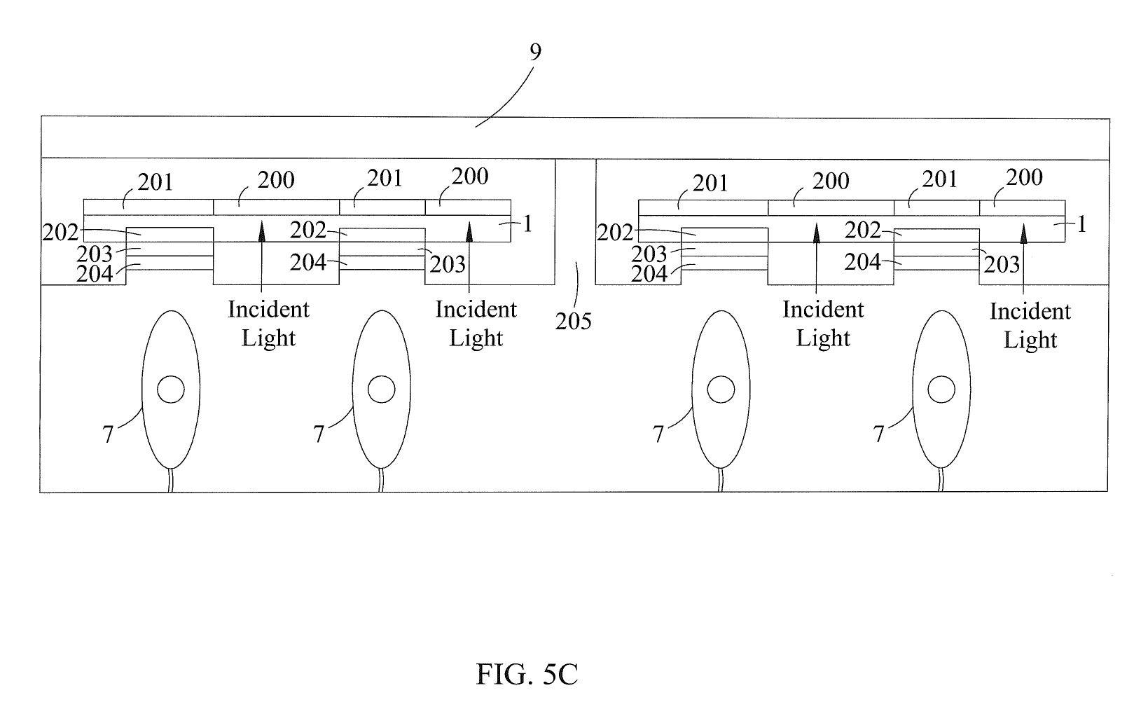

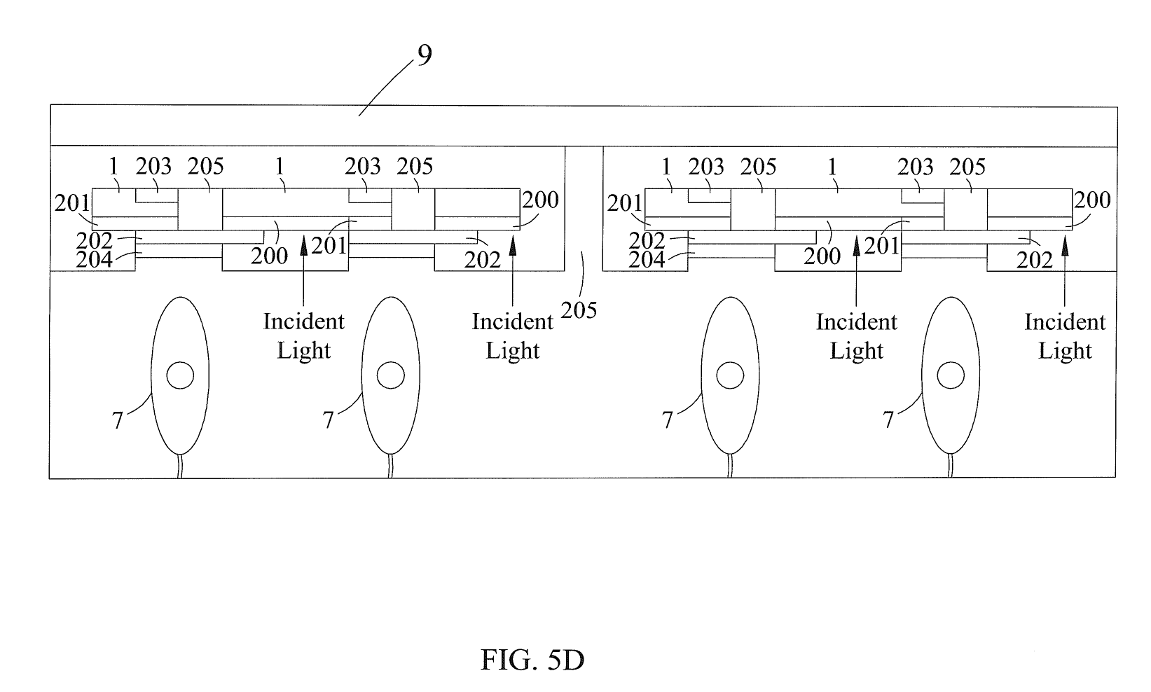

With reference to FIGS. 5A and 5B for schematic views of epi-retina stimulation apparatuses in accordance with a first preferred embodiment and a second preferred embodiment of the present invention respectively, the type of retina stimulation apparatuses 5 used in these preferred embodiments of the present invention is called epi-retina stimulation apparatus that uses the stimulating electrode 202 for connecting a ganglion cell 6 of the retina, and the photosensors 200 and signal processing and driving units 201 are disposed on the same side or a different side of the stimulating electrode 202 on a middle layer 1, wherein the middle layer 1 is preferably an oxide layer such as silicon oxide (SiO2). In the first preferred embodiment, the stimulating electrode 202, and the electrically conductive layer 203 (preferably aluminum) and selectively including a tissue glue 204 are disposed on a side of the middle layer 1 in sequence, and the photosensor 200 and the signal processing and driving unit 201 are disposed on the other side of the middle layer 1. In the second preferred embodiment, the photosensor 200, the signal processing and driving unit 201 and the stimulating electrode 202 are disposed on the middle layer 1, wherein a tissue glue 204 is selectively disposed on the stimulating electrode 202, and the electrically conductive layer 203 (preferably aluminum) and the perforation hole 205 are preferably disposed adjacent to the middle layer 1. This arrangement has the advantages of being able to receive a light from the rear lateral side of the retina stimulation apparatus 5 by a larger area (or the whole area), and the photosensor 200 can be installed under the stimulating electrode 202 but it should not cover the signal processing and driving unit 201.

With reference to FIGS. 5C and 5D for schematic views of sub-retina stimulation apparatuses in accordance with a first preferred embodiment and a second preferred embodiment of the present invention respectively, the type of retina stimulation apparatuses 5 used in these preferred embodiments of the present invention is called a sub-retina stimulation apparatus that uses the stimulating electrode 202 for connecting a bipolar cell 7 of the retina. The difference between the sub-retina stimulation apparatus and the epi-retina stimulation apparatus resides on that the sub-retina stimulation apparatus is installed between the bipolar cell 7 and the rods and cones 9. Same as the epi-retina stimulation apparatus, the photosensor 200 and the signal processing and driving unit 201 of the sub-retina stimulation apparatus are disposed on the same side or different sides of the stimulating electrode 202 on the middle layer 1. In addition, the guard ring is installed at a position that will not block the light incident to the photosensor 200, and the perforation hole 205 provides a passage for passing human fluid (liquid or gas) such as oxygen gas through the space between both sides of the retina stimulation apparatus to enhance the adaptability of this device for human body.

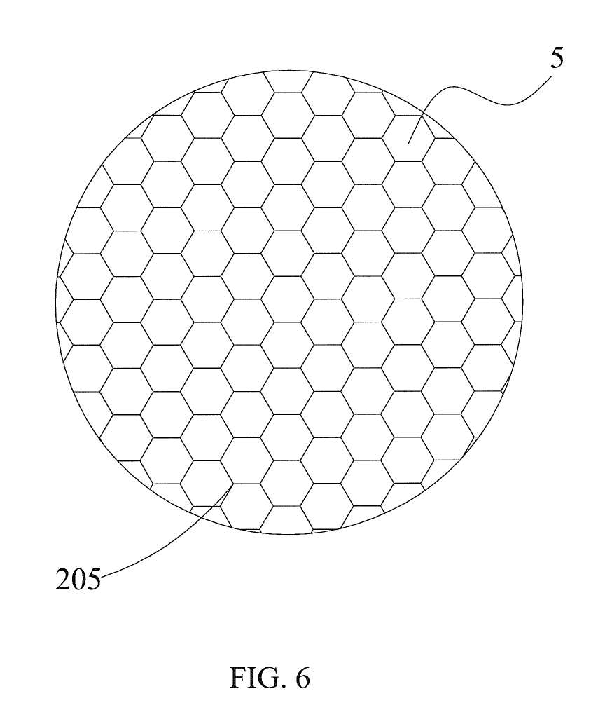

With reference to FIG. 6 for a schematic view of installing a plurality of retina stimulation apparatuses of the present invention, if the eyeball curvature is too large and exceeds the curvature of the flexible package, then the pixel units can be divided appropriately into a hexagonal retina stimulation apparatus, and a plurality of hexagonal retina stimulation apparatuses are combined to match with the eyeball curvature. The combination of a plurality of hexagonal retina stimulation apparatuses has the advantage of forming a passage for exchanging liquids at the boundary or the joint of the hexagonal retina stimulation apparatus. In other words, the perforation hole 205 facilitates the flow and exchange of liquid (body fluid) or gas such as oxygen gas.

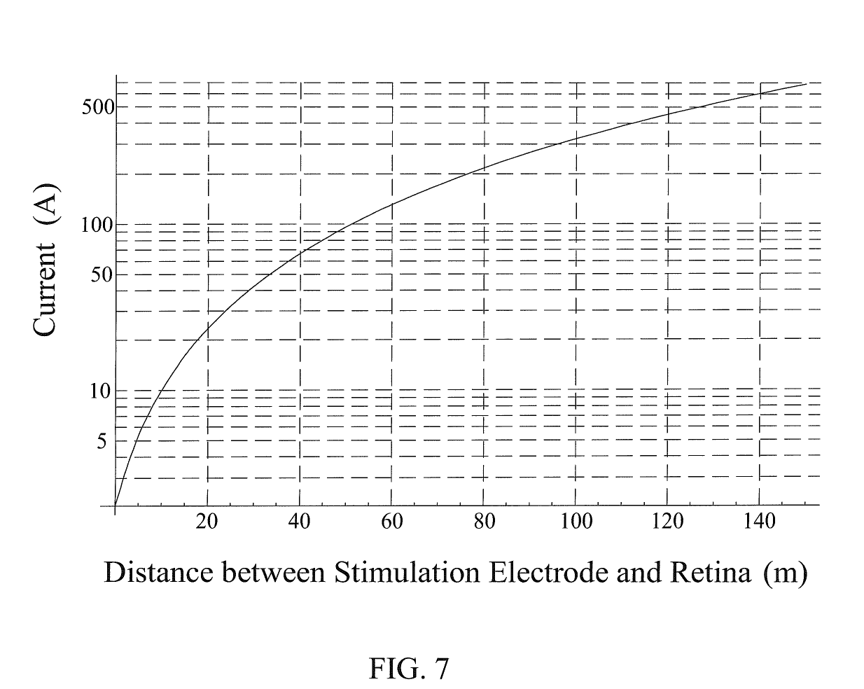

With reference to FIG. 7 for a graph of a stimulation current versus a distance of a retina from a stimulation electrode of a retina stimulation apparatus of the present invention, the transverse axis represents the distance of the retina stimulation apparatus from the retina, and the vertical axis represents the stimulation current of the retina stimulation apparatus. Since the present invention adopts a flexible substrate, therefore the substrate can be attached onto the eyeball surface to reduce the distance between the retina stimulation apparatus and the retina. Therefore, the retina stimulation apparatus of the present invention can reduce the stimulation current effectively to protect each cell.

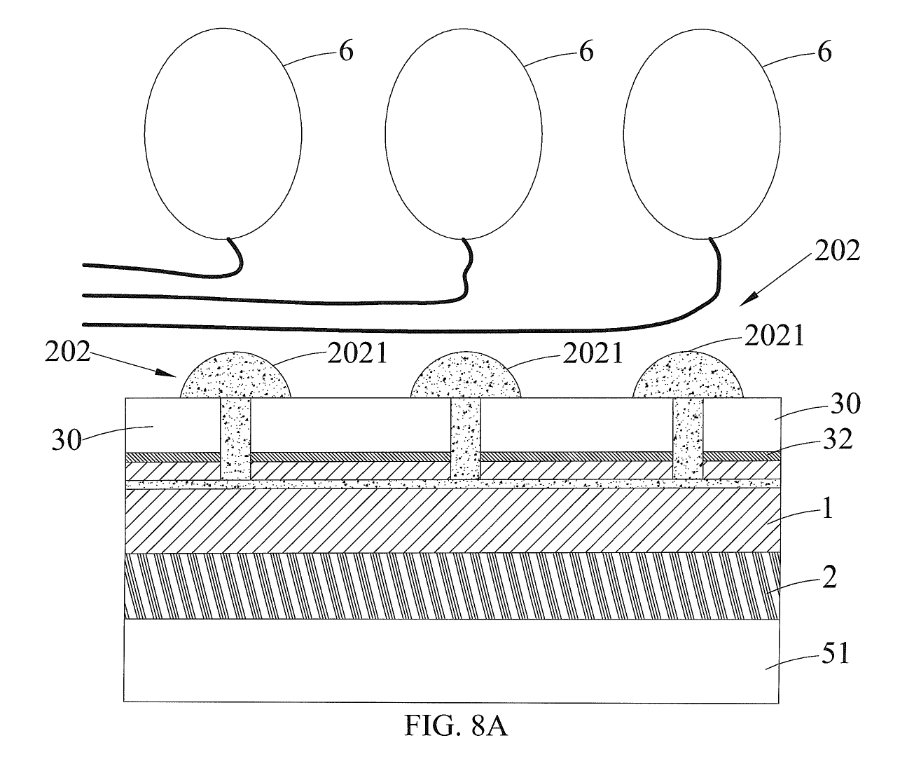

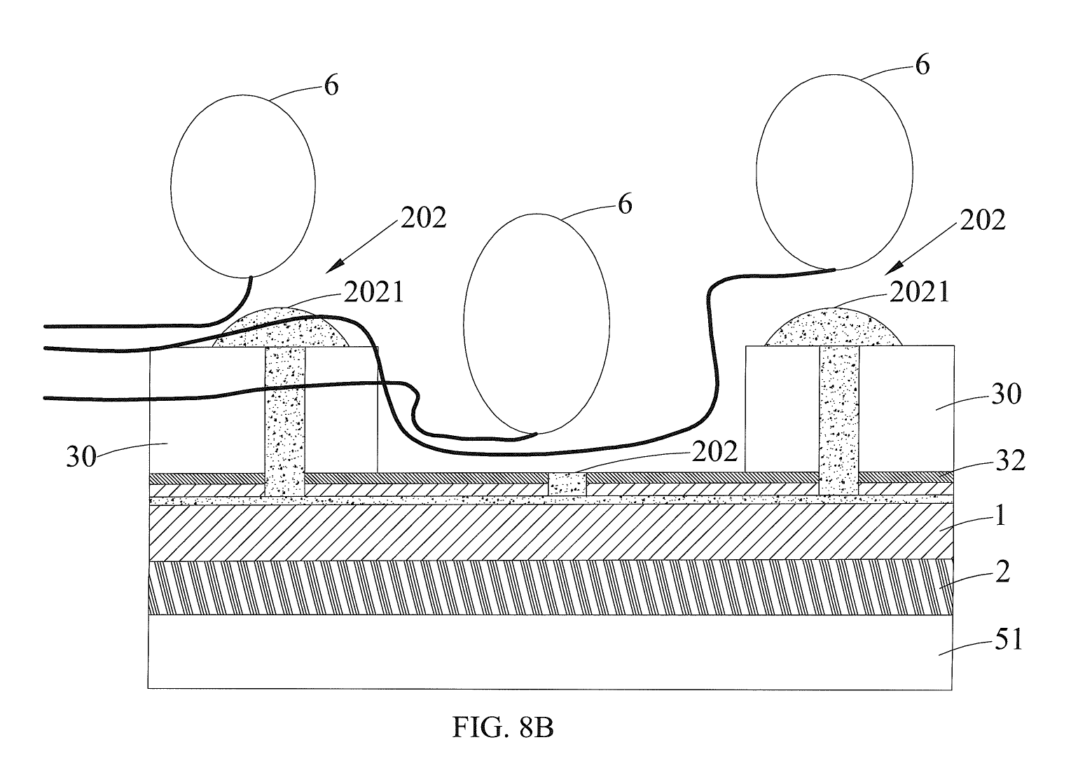

With reference to FIG. 8A for a schematic view of an epi-retina stimulation apparatus in accordance with a third preferred embodiment of the present invention, the epi-retina stimulation apparatus 5 comprises a flexible package 51, a pixel unit 20, a middle layer 1, a first biocompatible material layer 30, a barrier layer 32 and a stimulating electrode 202. In FIG. 8A, this preferred embodiment is characterized in that the stimulating electrode 202 is not disposed on the same plane of the pixel units 20, and the stimulating electrode 202 are erected in an umbrella shape to stimulate each ganglion cell 6, and the umbrella-shaped stimulating electrodes 202 includes a protrusion 2021. Therefore, the signal conductive wire connected to the bottom of the stimulating electrode 202 will not be in a direct contact with a ganglion cell 6 to avoid causing a wrong coupling or stimulation by mistake. With reference to FIG. 8B for another schematic view of an epi-retina stimulation apparatus in accordance with a third preferred embodiment of the present invention, the difference of this preferred embodiment from the preferred embodiment as shown in FIG. 8A resides on that each stimulating electrode 202 has a protrusion 2021 formed on a different plane. Therefore, this preferred embodiment can stimulate a portion or even a single ganglion cell 6 more precisely to achieve the effect of reducing the sensed light signal to produce a correct screen.

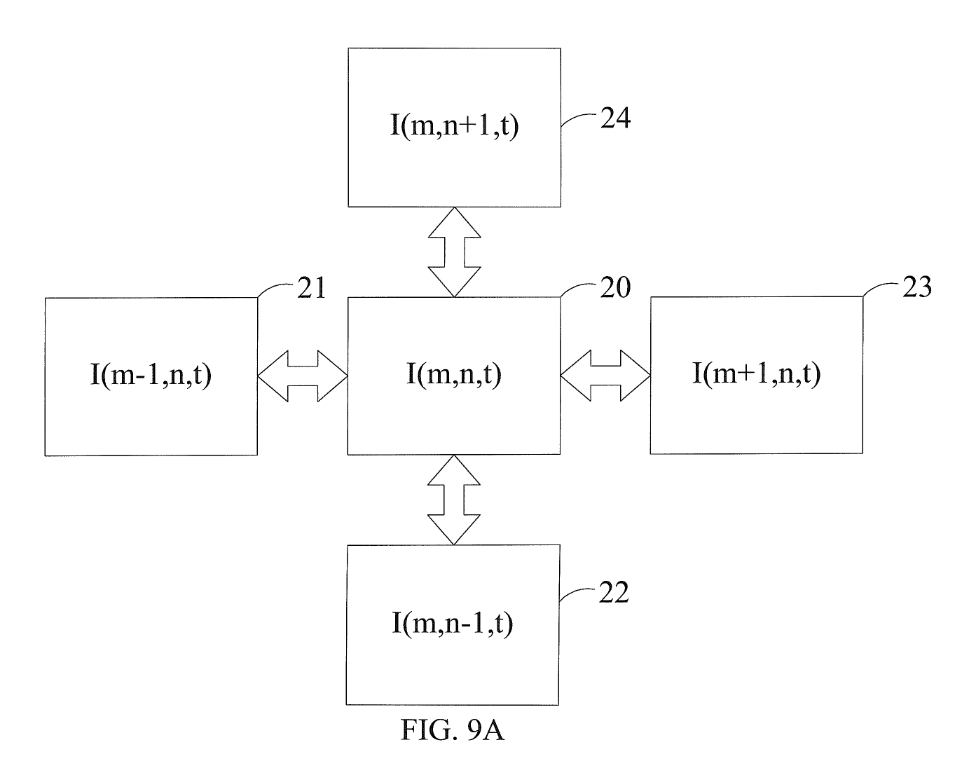

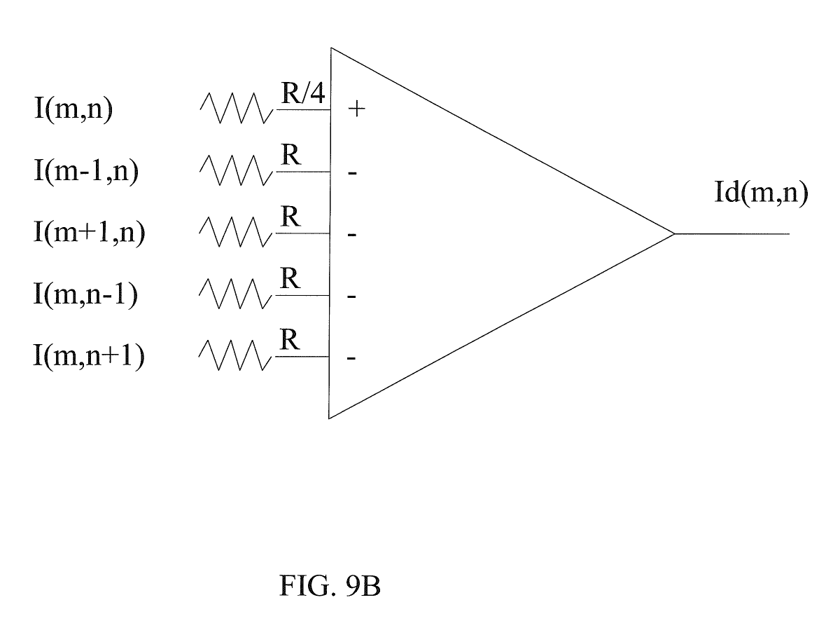

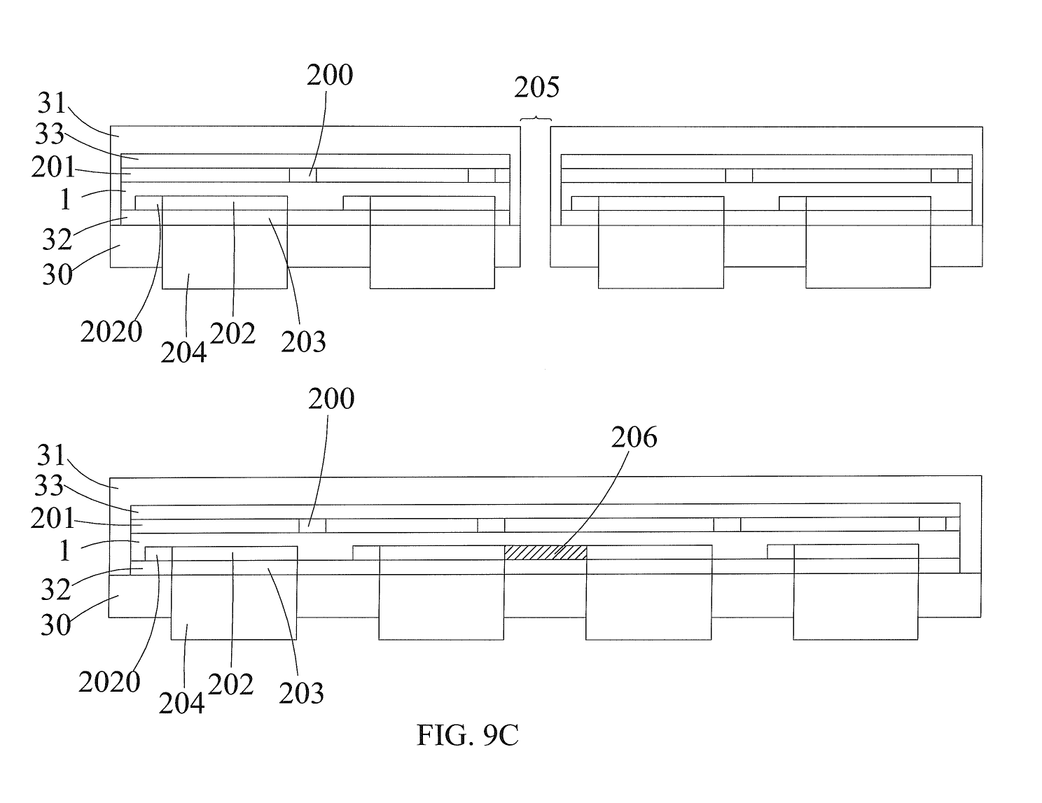

With reference to FIGS. 9A and 9B for a schematic view and a circuit diagram of adjusting the brightness of a background light by a signal processing and driving unit of the present invention respectively, a portion of the pixel array as shown in FIG. 9A includes a plurality pixel units 20, 21, 22, 23, 24. In this preferred embodiment, data are exchanged among the pixel units 20, 21, 22, 23, 24, and the data are exchanged by connecting the conductive wire 206 to different pixel units 20, 21, 22, 23, 24. In FIG. 9C, the pixel unit 20 at the middle is used as an example for illustrating the present invention, the pixel unit 20 can receive the light sensing data of other pixel units 21, 22, 23, 24, and the same processing procedure as shown in FIG. 9B takes place to output a current to stimulate a retinal cell to achieve the effect of adjusting the brightness of a background light, and a differential process is performed for the average value of the pixel units 21, 22, 23, 24 with the pixel unit 20, but the present invention is not limited to such arrangement only. The signal is used for adjusting the background brightness to enhance the resolution and recognition rate of image effectively. It is noteworthy to point out that the arrangement of pixel units is not limited to a square shape only, but the pixel units can be arranged into a hexagonal shape, or any pattern of pixel units closely disposed adjacent to one another.

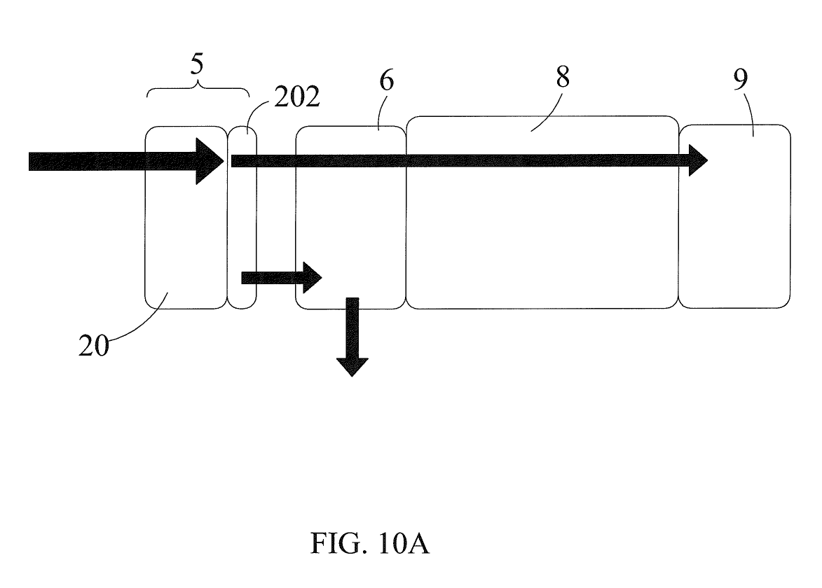

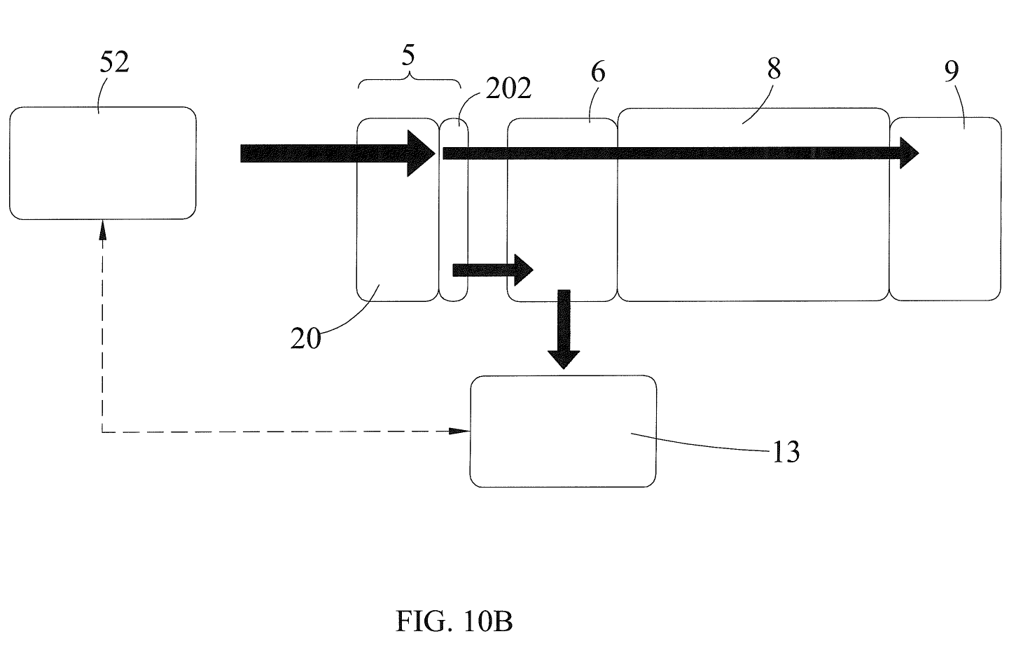

With reference to FIG. 10A for a schematic view of an epi-retina stimulation apparatus in accordance with a fourth preferred embodiment of the present invention, the epi-retina stimulation apparatus 5 comprises a pixel unit 20 and a stimulating electrode 202. The stimulating electrode 202 is disposed opposite to a ganglion cell 6 and provided for stimulating the cell. The ganglion cell 6 further includes a neural network 8 and rods and cones 9, wherein the neural network 8 includes different kinds of cells such as bipolar cells. In a normal human eye structure, an incident light is passed through the aforementioned ganglion cell 6, neural network 8 and rods and cones 9 and sensed and returned to the ganglion cell 6. However, the neural network 8 of some patients with a degenerated retina may become deteriorated, and the rods and cones 9 may be dead already, so that the epi-retina stimulation apparatus 5 of the present invention generates a stimulation signal to stimulate the ganglion cell 6 to produce a vision after receiving the incident light. Since the present invention adopts a very thin and soft substrate, therefore the epi-retina stimulation apparatus 5 can be attached completely onto the ganglion cell 6 to effectively reduce the required power for generating the stimulation signal. Due to the very small thickness, light can be passed through the retina stimulation apparatus 5 and installed on the ganglion cell 6. The invention is not limited to the arrangement of installing the apparatus 5 under the rods and cones 9 only. As shown in FIG. 10B, the present invention further comprises a remote control device 52 for receiving a feedback control signal transmitted from a human cerebral cortex 13, and controlling or adjusting each pixel unit 20 according to the feedback control signal for finely tuning the power of each stimulating electrode 202 to stimulate the retina (namely, brightness adjustment) to achieve the effect of a correct display. In addition, the remote control device 52 and the pixel unit 20 and human cerebral cortex 13 can be connected by an optical communication, a radio frequency communication or a wireless communication, but the invention is not limited to such arrangements only.

The retina stimulation apparatus of the present invention provides the flexible substrate matched with the eyeball curvature to reduce the current of stimulating the retina and prevent damaging the cells, and also successfully integrates the photosensor, driving device and stimulating electrode in a pixel unit to overcome the problem of poor sensing and stimulating effects.

The present invention has been described with some preferred embodiments thereof and it is understood that many changes and modifications in the described embodiments can be carried out without departing from the scope and the spirit of the invention that is intended to be limited only by the appended claims.

What is claimed is:

1. A retina stimulation apparatus, comprising:
a plurality of pixel units, comprising:
a photosensor for sensing an incident light and generating a sensing signal;
a signal processing and driving unit for receiving and processing the sensing signal to generate an electric stimulation waveform; and
a stimulating electrode for receiving the electric stimulation waveform to generate a corresponding stimulation current to stimulate a retinal cell; and
a flexible package for carrying and covering the pixel unit.

2. The retina stimulation apparatus of claim 1, wherein the retina stimulation apparatus is installed on a ganglion cell, and the photosensors and the signal processing and driving units are installed on a different side of the stimulating electrodes on a middle layer.

3. The retina stimulation apparatus of claim 1, wherein the retina stimulation apparatus is installed on a ganglion cell, and the photosensors and the signal processing and driving units are installed on the same side of the stimulating electrodes on a middle layer.

4. The retina stimulation apparatus of claim 1, wherein the retina stimulation apparatus is installed between a bipolar cell and a rod and cone, and the photosensors and the signal processing and driving units are installed on a different side of the stimulating electrodes on a middle layer.

5. The retina stimulation apparatus of claim 1, wherein the retina stimulation apparatus is installed between a bipolar cell and a rod and cone, and the photosensors and the signal processing and driving units are installed on the same side of the stimulating electrodes on a middle layer.

6. The retina stimulation apparatus of claim 1, further comprising a power supply module coupled to the pixel unit for supplying electric power to the pixel units after the power supply module is charged.

7. The retina stimulation apparatus of claim 1, further comprising a perforation hole formed between the pixel units includes for allowing air or a tissue liquid to flow through both upper and lower sides of the retina stimulation apparatus.

8. The retina stimulation apparatus of claim 1, wherein each of the signal processing and driving units is electrically coupled by a conductive wire for exchanging each of the sensing signals to adjust the intensity of a background light.

9. The retina stimulation apparatus of claim 1, further comprising a guard ring installed under each of the stimulating electrodes for regionally stimulating the retinal cell.

10. The retina stimulation apparatus of claim 1, wherein each the signal processing and driving unit further comprises a sensing circuit for detecting the mode of the retinal cell, and each signal processing and driving unit controls the stimulating electrode to stimulate the retinal cell according to the mode of the retinal cell.

11. The retina stimulation apparatus of claim 1, wherein the stimulating electrodes are convex umbrella-shaped stimulating electrodes.

12. The retina stimulation apparatus of claim 11, wherein each convex umbrella-shaped stimulating electrode includes a plurality of protrusions disposed on the same plane.

13. The retina stimulation apparatus of claim 1, wherein the flexible package is made of a biocompatible material.

14. The retina stimulation apparatus of claim 13, wherein the biocompatible material is one selected from the collection of polyimide, poly(dimethyl siloxane) (PDMS) and parylene.

15. The retina stimulation apparatus of claim 1, further comprising a remote control device coupled to the pixel units by a wireless communication method for controlling the pixel units from a long distance.

16. The retina stimulation apparatus of claim 15, wherein the wireless communication method for connecting the remote control device with the pixel units is an optical communication, a radio frequency communication or a wireless communication.

17. A manufacturing method of a retina stimulation apparatus, comprising the steps of:
providing a substrate, and integrating a plurality of signal processing and driving units, a plurality of photosensors and a plurality of stimulating electrodes to form a plurality of pixel units on the substrate;
forming an electrically conductive layer on the stimulating electrodes, a first barrier layer on the other area of the pixel units, and a first biocompatible material layer on the first barrier layer;
forming a first holding substrate on the first barrier layer;
removing the substrate to expose the signal processing and driving units and the photosensors;
forming a second barrier layer on the exposed signal processing and driving units and the photosensors;
forming a perforation hole between two of the pixel units;
forming a second biocompatible material layer on the second barrier layer, and covering the first biocompatible material layer and the second biocompatible material layer on each of the pixel units;
forming a second holding substrate on the second biocompatible material layer;
removing a portion of the first biocompatible material layer to expose the electrically conductive layer from the stimulating electrodes; and
removing the second holding substrate.

18. The manufacturing method of a retina stimulation apparatus as recited in claim 17, further comprising a conductive wire installed between the signal processing and driving units for exchanging the sensing signals to adjust the intensity of a background light.

19. The manufacturing method of a retina stimulation apparatus as recited in claim 17, further comprising a power supply module coupled to the plurality of pixel units, for supplying electric power to the plurality of pixel units after the power supply module is charged.

20. The manufacturing method of a retina stimulation apparatus as recited in claim 17, wherein the photosensor is provided for sensing an incident light to generate a sensing signal, and the signal processing and driving unit is provided for receiving and processing the sensing signal to generate a corresponding electric stimulation waveform, and the stimulating electrode is provided for receiving the electric stimulation waveform to generate a corresponding stimulation current to stimulate a retinal cell.

21. The manufacturing method of a retina stimulation apparatus as recited in claim 17, further comprising a guard ring installed under the stimulating electrodes for regionally stimulating the retinal cell.

22. The manufacturing method of a retina stimulation apparatus as recited in claim 17, further comprising a sensing circuit installed in each of the signal processing and driving units for detecting the mode of the retinal cell, and each of the signal processing and driving units controls the stimulating electrode to stimulate the retinal cell according to the mode of the retinal cell.

23. The manufacturing method of a retina stimulation apparatus as recited in claim 17, wherein the stimulating electrodes are convex umbrella-shaped stimulating electrodes.

24. The manufacturing method of a retina stimulation apparatus as recited in claim 23, wherein each of the convex umbrella-shaped stimulating electrodes includes a plurality of protrusions not formed on the same plane.

25. The manufacturing method of a retina stimulation apparatus as recited in claim 17, further comprising a remote control device coupled to the pixel units by a wireless communication method and provided for controlling the pixel units from a long distance.

26. The manufacturing method of a retina stimulation apparatus as recited in claim 25, wherein the wireless communication method for connecting the remote control device with the pixel units is an optical communication, a radio frequency communication or a wireless communication.

27. The manufacturing method of a retina stimulation apparatus as recited in claim 17, wherein the first biocompatible material layer and the second biocompatible material layer are made of a flexible material.

28. The manufacturing method of a retina stimulation apparatus as recited in claim 27, wherein the flexible material is one selected from the collection of polyimide, poly(dimethyl siloxane) (PDMS) and parylene.

29. The manufacturing method of a retina stimulation apparatus as recited in claim 17, wherein the first barrier layer and the second barrier layer are made of silicon carbide (SiC) or diamond like carbon film (DLC film).
